(19) United States
(12) Patent Application Publication
(10) Pub. No.: US 2024/0180420 A1
(43) Pub. Date: Jun. 6, 2024

(54) A METHOD AND DEVICE FOR EARLY DETECTION OF AN OCULAR DISEASE

(71) Applicant: Essilor International, Charenton-Le-Pont (FR)

(72) Inventor: Guillaume GIRAUDET, Charenton-Le-Pont (FR)

(73) Assignee: Essilor International, Charenton-Le-Pont (FR)

(21) Appl. No.: 18/553,477

(22) PCT Filed: Feb. 9, 2022
(86) PCT No.: PCT/EP2022/053129

§ 371 Date: Sep. 29, 2023

(30) Foreign Application Priority Data
Mar. 30, 2021 (EP) 21305398.6

(51) Int. Cl. A61B 3/10; A61B 5/00; A61B 5/11

(52) U.S. Cl. CPC A61B 3/10; A61B 5/1114; A61B 5/7275; A61B 5/7278

(57) ABSTRACT

A method for obtaining information related to a purported evolution of a visual field of an individual that may be a forerunner of an eye disease. The method includes providing a plurality of values of at least one parameter related to head movements, representing a reference head movement behavior, measuring a same at least one parameter related to head movements of the individual in daily life, during a predetermined measurement period, comparing the individual's head movement behavior with the reference head movement behavior, in case of detecting a difference between both head movement behaviors, checking whether there is any specific reason in the individual's daily life explaining that difference and, if there is no specific reason, obtaining, on the basis of that difference, information related to a purported evolution of the visual field that may be a forerunner of an eye disease.

### FIELD OF THE DISCLOSURE

The present disclosure relates to a method and device for early detection of an ocular disease. More particularly, the present disclosure relates to a method and device for obtaining information related to a purported evolution of a visual field of an individual that may be a forerunner of an eye disease.

### BACKGROUND OF THE DISCLOSURE

The present disclosure may apply to any ocular disease causing peripheral i.e. side vision loss. Vision loss may be associated with numerous diseases, such as carotid artery diseases, cytomegalovirus retinitis, diabetic eye diseases, diabetic and/or myopic retinopathy, glaucoma, HIV/AIDS, idiopathic intracranial hypertension, ischemic optic neuropathy, low vision, macular degeneration, pigment dispersion syndrome, retinal artery occlusion, retinitis pigmentosa, Stickler syndrome, etc.

For simplification, by way of non-limiting example, in the following, only the example of glaucoma will be considered.

Glaucoma is one of the leading causes of irreversible blindness with a prevalence of 3.5% in the population aged 40-80 years. In 2013, the disease affected 64.3 million people worldwide and it is expected to increase to 111.8 million by 2040, according to the article by Y.- C. Tham et al. entitled “Global Prevalence of Glaucoma and Projections of Glaucoma Burden through 2040: A Systematic Review and Meta-Analysis”, Ophthalmology, vol. 121, issue 11, November 2014, Elsevier, pages 2081-2090.

Glaucoma causes morphological changes of the optic disk accompanied by visual field damage that is slowly progressing, with a time course of several years. The patient is often not aware of visual field loss in the early stages of the disease. The lack of symptoms up to advanced stages induces an undetection rate of about 50%, even in so-called “developed” countries. And the later the treatment is carried out, the greater and more permanent are the functional consequences.

Currently, the diagnosis of glaucoma generally relies on the assessment of intraocular pressure, as well as specific changes occurring in the visual field, the optic nerve head, and the retinal nerve fiber layer. Visual field is studied by means of automated perimetry. Perimetry is commonly considered the diagnostic gold-standard for glaucoma progression.

Yet, perimetry is limited by low diagnostic sensitivity in early stages of the disease and, being a psychophysical test, it is also influenced by learning, fatigue, eye movements, etc. Furthermore, clinical tests are performed by or at least under the supervision of eye doctors and optometrists. For those who do not have regular eye examination and/or do not have easy access to Eye Care Professionals (ECPs), the first consultation for glaucoma suspicion usually follows conscious perception by the patient of glaucoma-related vision symptoms or takes place even later. When symptoms are consciously experienced, the disease has been already there for a while and the treatment will require heavier dosage or even surgery.

Therefore, there is a need for earlier detection of glaucoma or any other ocular disease causing peripheral vision loss, before symptoms get noticeable by the patient.

Previous research in the art suggested using eye tracking systems to that end. However, eye tracking systems have a number of drawbacks in the field of autonomous smart eyeglasses products:
they consume a lot of energy, hence it is difficult to provide all-day-long eye movement recordings;
they are difficult to calibrate and the signal is easily lost when eyes make large saccades and/or look down, because of the eyelid lowering;
they are usually heavy and bulky, hence not very convenient in full-time wearing.

Document EP 3 075 315 A1 discloses a method for monitoring the visual behavior of a person.

Document EP 3 789 816 A1 discloses a device and method for mapping a visual scene onto a projection surface.

Document EP 3 461 394 A1 discloses a method and system for adapting the visual and/or visual-motor behavior of a person.

### SUMMARY OF THE DISCLOSURE

An object of the disclosure is to overcome the above-mentioned drawbacks of the prior art, in particular by providing behavioral data that constitute sensitive and selective markers of early onset of glaucoma or other ocular diseases causing peripheral vision loss.

To that end, the disclosure provides a method for obtaining information related to a purported evolution of a visual field of an individual, wherein it comprises:
providing a plurality of values of at least one parameter related to head movements, representing a reference head movement behavior;
measuring a same at least one parameter related to head movements of the individual in daily life, during a predetermined measurement period, so as to obtain a plurality of measurements of the at least one parameter, representing the individual's head movement behavior during the predetermined measurement period;
comparing the individual's head movement behavior during the predetermined measurement period with the reference head movement behavior;
in case of detecting a difference between the individual's head movement behavior during the predetermined measurement period and the reference head movement behavior:
   checking whether there is any specific reason in the individual's daily life explaining that difference;
if there is no such specific reason, obtaining, on the basis of that difference, information related to a purported evolution of the visual field.

Thus, based on the head movements of an individual in the individual's daily life and environment, by identifying head movements behavioral change, in natural vision conditions, the method according to the present disclosure makes it possible to improve the early detection of ocular diseases causing loss of peripheral vision, such as glaucoma, but also carotid artery diseases, cytomegalovirus retinitis, diabetic eye diseases, diabetic and/or myopic retinopathy, HIV/AIDS, idiopathic intracranial hypertension, ischemic optic neuropathy, low vision, macular degeneration, pigment dispersion syndrome, retinal artery occlusion, retinitis pigmentosa, Stickler syndrome, etc.

Indeed, even if monocular and early binocular visual field defects do not reach conscious perception, they induce subtle visuomotor changes that can be detected by the method according to the present disclosure.

In an embodiment, the step of providing comprises generating a statistical model of head movements taking account of at least one datum related to the individual.

In other words, thanks to the large amount of data that may be gathered by the method according to the present disclosure, statistical models of “usual” head movements can be generated for each individual.

In that embodiment, according to a particular possible characteristic, the step of generating comprises:
measuring the at least one parameter related to head movements of the individual in daily life, during a reference time period equal to a predetermined number of times the measurement period, so as to obtain a plurality of measurements of the at least one parameter, representing the individual's head movement behavior during the reference time period; and
computing, at an end of the reference time period, a prevalence of the at least one parameter related to head movements of the individual in daily life during the reference time period, to be used as the statistical model for the individual;

wherein the method further comprises computing, at an end of the predetermined measurement period, a prevalence of the at least one parameter related to head movements of the individual in daily life during the predetermined measurement period; and wherein the step of comparing comprises comparing the prevalence computed at the end of the predetermined measurement period with the prevalence computed at the end of the reference time period.

In that embodiment, according to a particular possible characteristic, the step of generating comprises determining the reference time period, by:
(i) measuring, during a first time period equal to the measurement period, the at least one parameter related to head movements of the individual in daily life, so as to obtain a first plurality of measurements of the at least one parameter;
(ii) measuring, during a second time period equal to the measurement period, the at least one parameter related to head movements of the individual in daily life, so as to obtain a second plurality of measurements of the at least one parameter;
(iii) determining whether there are differences considered as significant between the first and second pluralities of measurements;
(iv) in case there is no difference considered as significant between the first and second pluralities of measurements, determining the reference time period as the measurement period;
(v) in case there are differences considered as significant between the first and second pluralities of measurements:
   (a) measuring, during an additional time period equal to the measurement period, the at least one parameter related to head movements of the individual in daily life, so as to obtain an additional plurality of measurements;
   (b) determining whether there are differences considered as significant between, on the one hand, cumulated pluralities of measurements obtained at steps (i) and (ii) and, on the other hand, the additional plurality of measurements; and
   (c) iterating step (a) during further time periods, each equal to the measurement period, as long as there are differences considered as significant between pluralities of measurements used at step (b), so as to eventually determine the reference time period as an accumulation of time periods of steps (i), (ii), (a) and (c).

In an embodiment, the step of providing comprises using as the reference head movement behavior an average model of head movements of a human being.

In an embodiment, the at least one parameter is taken in a group comprising a rotation amplitude of each one of the head movements, a rotation speed of each one of the head movements and a variance of rotation speed of the head movements.

In an embodiment, the measurement period is one month.

In an embodiment, the method further comprises, if there is no specific reason in the individual's daily life explaining the difference between the individual's head movement behavior during the predetermined measurement period and the reference head movement behavior, generating an alert advising the individual to have eyes checked by an eye care professional.

In that embodiment, according to a particular possible characteristic, the alert generating is performed only if the difference between the individual's head movement behavior during the predetermined measurement period and the reference head movement behavior exceeds a predetermined threshold. In that embodiment, according to a particular possible characteristic, the method further comprises dispatching the alert through a mobile communication device.

In an embodiment, the method further comprises, in case of detecting a difference between the individual's head movement behavior during the predetermined measurement period and the reference head movement behavior, assigning a different weight to the difference depending on the direction of head movements.

In an embodiment, the detecting comprises determining whether head movements have changed in one hemi-field and not in another hemi-field.

In an embodiment, the method further comprises:
dividing an area comprising all possible head movements into a plurality of subareas, each of the subareas comprising a different predetermined percentage of all head movements for which the at least one parameter is measured during the measuring; and
performing for each of the subareas the step of comparing the individual's head movement behavior during the predetermined measurement period with the reference head movement behavior.

In an embodiment, the measuring is continuous.

To the same end as mentioned above, the present disclosure also provides a device for obtaining information related to a purported evolution of a visual field of an individual, wherein it comprises:
a unit for obtaining a plurality of values of at least one parameter related to head movements, representing a reference head movement behavior;
at least one sensor configured for measuring a same at least one parameter related to head movements of the individual in daily life, during a predetermined measurement period, so as to obtain a plurality of measurements of the at least one parameter, representing the individual's head movement behavior during the predetermined measurement period;
at least one processor configured for:
comparing the individual's head movement behavior during the predetermined measurement period with the reference head movement behavior;
in case of detecting a difference between the individual's head movement behavior during the predetermined measurement period and the reference head movement behavior:
   checking whether there is any specific reason in the individual's daily life explaining that difference;
if there is no such specific reason, obtaining, on the basis of that difference, information related to a purported evolution of the visual field.

In an embodiment, the device is a pair of smart eyeglasses and the at least one sensor comprises at least one gyroscope and at least one accelerometer.

To the same end as mentioned above, the present disclosure further provides a computer program product comprising one or more sequences of instructions that are accessible to a processor and that, when executed by the processor, cause the processor to:
obtain a plurality of values of at least one parameter related to head movements, representing a reference head movement behavior;
receive a plurality of measurements of a same at least one parameter related to head movements of an individual in daily life, during a predetermined measurement period, representing the individual's head movement behavior during the predetermined measurement period;
compare the individual's head movement behavior during the predetermined measurement period with the reference head movement behavior;
in case of detecting a difference between the individual's head movement behavior during the predetermined measurement period and the reference head movement behavior:
   check whether there is any specific reason in the individual's daily life explaining that difference;
if there is no such specific reason, obtain, on the basis of that difference, information related to a purported evolution of a visual field of the individual.

To the same end as mentioned above, the present disclosure further provides a non-transitory information storage medium, wherein it stores one or more sequences of instructions that are accessible to a processor and that, when executed by the processor, cause the processor to:
obtain a plurality of values of at least one parameter related to head movements, representing a reference head movement behavior;
receive a plurality of measurements of a same at least one parameter related to head movements of an individual in daily life, during a predetermined measurement period, representing the individual's head movement behavior during the predetermined measurement period;
compare the individual's head movement behavior during the predetermined measurement period with the reference head movement behavior;
in case of detecting a difference between the individual's head movement behavior during the predetermined measurement period and the reference head movement behavior:
   check whether there is any specific reason in the individual's daily life explaining that difference;
if there is no such specific reason, obtain, on the basis of that difference, information related to a purported evolution of a visual field of the individual.

As the advantages of the device, of the computer program product and of the computer-readable storage medium are similar to those of the method, they are not repeated here.

The computer program product and the computer-readable storage medium are advantageously configured for executing the method in any of its execution modes.

### BRIEF DESCRIPTION OF THE DRAWINGS

For a more complete understanding of the description provided herein and the advantages thereof, reference is now made to the brief descriptions below, taken in connection with the accompanying drawings and detailed description, wherein like reference numerals represent like parts.

FIG. 1 is a flow diagram showing steps of a method according to the present disclosure, in a particular embodiment.

FIGS. 2 and 3 are graphs illustrating non-limiting examples of the outcome of head movement measurements made during a predetermined measurement period, in a particular embodiment of a method according to the present disclosure.

FIG. 4 is a map representing a normal binocular visual field of a given individual and showing overlaps and differences between left and right monocular visual fields.

FIG. 5 is a graph illustrating an evolution of head movements with respect to those represented in the example of FIG. 3, according to head movement measurements made during a predetermined measurement period, in a particular embodiment of a method according to the present disclosure.

FIG. 6 is a schematic view of a device according to the present disclosure, in a particular embodiment.

### DETAILED DESCRIPTION OF THE DISCLOSURE

In the description which follows, although making and using various embodiments are discussed in detail below, it should be appreciated that as described herein are provided many inventive concepts that may embodied in a wide variety of contexts. Embodiments discussed herein are merely representative and do not limit the scope of the disclosure. It will also be obvious to one skilled in the art that all the technical features that are defined relative to a process can be transposed, individually or in combination, to a device and conversely, all the technical features relative to a device can be transposed, individually or in combination, to a process and the technical features of the different embodiments may be exchanged or combined with the features of other embodiments.

The terms “comprise” (and any grammatical variation thereof, such as “comprises” and “comprising”), “have” (and any grammatical variation thereof, such as “has” and “having”), “contain” (and any grammatical variation thereof, such as “contains” and “containing”), and “include” (and any grammatical variation thereof such as “includes” and “including”) are open-ended linking verbs. They are used to specify the presence of stated features, integers, steps or components or groups thereof, but do not preclude the presence or addition of one or more other features, integers, steps or components or groups thereof. As a result, a method, or a step in a method, that “comprises”, “has”, “contains”, or “includes” one or more steps or elements possesses those one or more steps or elements, but is not limited to possessing only those one or more steps or elements.

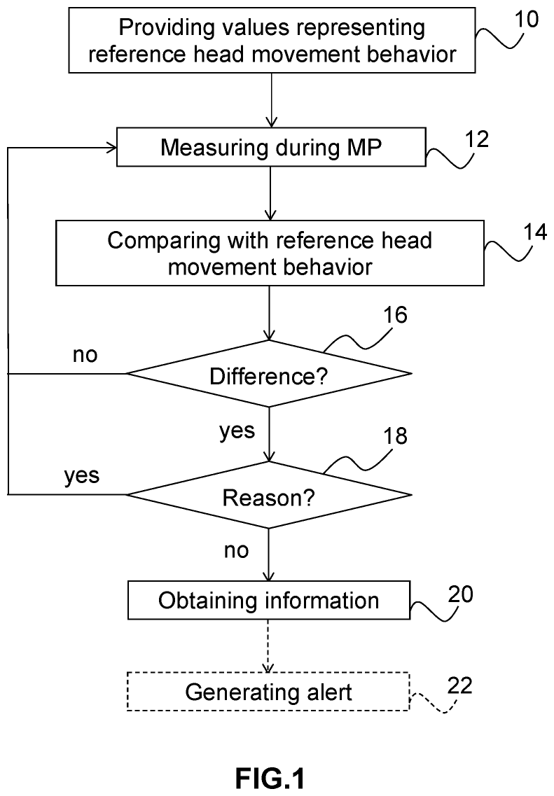

The flow diagram of FIG. 1 shows steps of a method according to the present disclosure in a particular embodiment. The method is for obtaining information related to a purported evolution of a visual field of an individual.

That purported evolution may be a forerunner of an eye disease. By way of non-limiting example, the eye disease may be a disease that causes damages to peripheral i.e. side vision, such as carotid artery diseases, cytomegalovirus retinitis, diabetic eye diseases, diabetic and/or myopic retinopathy, glaucoma, HIV/AIDS, idiopathic intracranial hypertension, ischemic optic neuropathy, low vision, macular degeneration, pigment dispersion syndrome, retinal artery occlusion, retinitis pigmentosa, Stickler syndrome, etc.

As shown in FIG. 1, the method comprises a step 10 of providing a plurality of values of at least one parameter related to head movements, representing a reference head movement behavior, also referred to hereafter as the “baseline situation”.

In an embodiment, an average model of head movements of a human being may be used as the baseline situation.

As a variant, the baseline situation may be established based on the considered individual's daily tasks and activities, in which case the providing step 10 comprises generating a statistical model of head movements taking account of at least one datum related to the considered individual. The at least one datum may be the above-mentioned at least one parameter, referred to hereafter as “the parameter(s)”. The parameter(s) reflect(s) the tasks and activities of the individual.

The parameter(s) may be the rotation amplitude of each one of the head movements, in predetermined directions. For instance, the left and right directions and the up and down directions may be considered.

The parameter(s) may also be the rotation speed of each one of the head movements.

It may also be the variance of that rotation speed.

Any of the above parameters is interesting because, when peripheral vision sensitivity lowers with glaucoma, peripheral targets are perceived later and reflex high speed head movements are more frequent.

The parameter(s) may be measured. In such an embodiment, the baseline situation may be set for instance after a certain period of measurement.

In an embodiment where the baseline situation is established based on the considered individual's daily tasks and activities, the measuring of the parameter(s) may be continuous.

Let us define MP as the minimal period during which head movements are measured and possibly, but not necessarily, recorded and/or stored, in order to provide a pattern of head behavior.

In the above-mentioned variant where the baseline situation is established based on the considered individual's daily tasks and activities, the step of generating the statistical model may comprise a step of measuring the parameter(s) related to head movements of the considered individual in daily life during a reference time period, hereafter referred to as the “baseline arbitrary period”, so as to obtain a plurality of measurements of the parameter(s), representing the individual's head movement behavior during the baseline arbitrary period.

At the end of the baseline arbitrary period, the prevalence of the parameter(s) related to the individual's head movements in daily life during the baseline arbitrary period is computed, in order to be used as the statistical model for that individual.

The baseline arbitrary period may be set at a predefined number of times MP.

By way of non-limiting example, MP may be one day, one week, or preferably, one month.

By way of non-limiting example, if MP is one month, the baseline arbitrary period may be one year, i.e. 12 MP, considering that, after measuring head movements during one year, all the considered individual's usual daily tasks and activities have been considered in various environments and situations, e.g. different seasons, work, vacation, etc.

Nevertheless, if the considered individual is of the opinion that a shorter time period is sufficient to be representative of all of his or her usual tasks and activities, the baseline arbitrary period may be set at N times MP where N is a non-null integer strictly lower than 12, for example 10 MP, 6 MP or even less.

As a variant, the baseline situation may be computed from the gradual time accrual of MPs over a certain period of time. In other words, in that variant, the following steps may be carried out:
(i) measuring, during a first time period equal to MP, the parameter(s) related to head movements of the individual in daily life, so as to obtain a first plurality of measurements of the parameter(s);
(ii) measuring, during a second time period equal to MP, the parameter(s) related to head movements of the individual in daily life, so as to obtain a second plurality of measurements of the parameter(s);
(iii) determining whether there are differences considered as significant between the first and second pluralities of measurements;
(iv) in case there is no difference considered as significant between said first and second pluralities of measurements, determining said reference time period as said measurement period;
(v) in case there are differences considered as significant between the first and second pluralities of measurements:
   (a) measuring, during an additional time period equal to MP, the parameter(s) related to head movements of the individual in daily life, so as to obtain an additional plurality of measurements;
   (b) determining whether there are differences considered as significant between, on the one hand, cumulated pluralities of measurements obtained at steps (i) and (ii) and, on the other hand, the additional plurality of measurements; and
   (c) iterating step (a) during further time periods, each equal to MP, as long as there are differences considered as significant between pluralities of measurements used at step (b), so as to eventually determine the baseline arbitrary period as an accumulation of time periods of steps (i), (ii), (a) and (c).

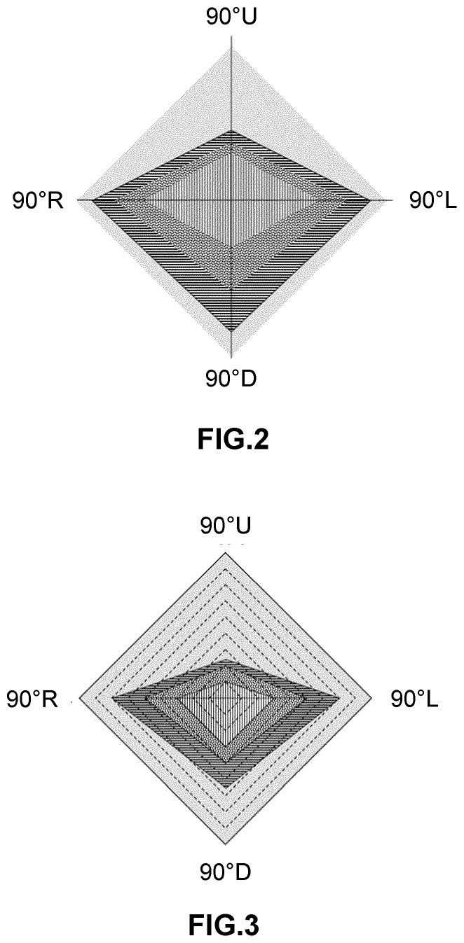

The graphs of FIGS. 2 and 3 show two different results of computation of the prevalence of amplitudes of head movements at the end of MP in non-limiting examples where MP lasts one month.

The left end of the diamond-shaped graphs represents a head movement having a rotation amplitude of 90 degrees on the left (“90ºL” on the drawing), the right end of the graphs represents a head movement having a rotation amplitude of 90 degrees on the right (“90° R”), the upper end represents a head movement having a rotation amplitude of 90 degrees up (“90° U”) and the lower end represents a head movement having a rotation amplitude of 90 degrees down (“90° D”).

The central hatched area represents 50% of the total amount of head movements done by the individual during MP. The central+middle hatched areas represent 85% of the head movements. The central+middle+outer hatched areas represent 100% of the head movements.

In FIG. 3, where inner diamond-shapes in dashed lines each represent steps of 10 degrees, it can be observed that 50% of head rotation amplitudes during MP are in the range of 30-30 degrees right-left and 10-30 degrees up-down, 85% of head rotation amplitudes during MP are in the range of 50-50 degrees right-left and 20-40 up-down and all the individual's head movements had rotation amplitudes in the range of 70-70 degrees right-left and 25-55 degrees up-down.

Referring back to FIG. 1, in order to obtain information related to a purported evolution of the individual's visual field that may be a forerunner of an eye disease, after establishing the baseline situation as a result of the providing step 10, the method according to the present disclosure further comprises a step 12 of measuring the same at least one parameter related to head movements of the considered individual in his or her daily life.

Having three areas representing respectively 50%, 85% and 100% of the total amount of head movements, as described above with reference to FIGS. 2 and 3, is only a non-limiting example.

In an embodiment, the method according to the present disclosure further comprises dividing the area comprising all possible head movements into a plurality of subareas, each of the subareas comprising a different predetermined percentage of all head movements for which the parameter(s) is/are measured during the measuring step 12. The areas may be located at different positions regarding both the horizontal and vertical head rotation movements in order to increase sensitivity of the method.

Like for the baseline situation, the parameter(s) is/are measured for the considered individual during the period MP, which is used this time as a predetermined measurement period, rather than as the baseline arbitrary period. As a result, a plurality of measurements of the parameter(s) is obtained, which represent the considered individual's head movement behavior during MP.

Like for the baseline situation, in an embodiment, the measuring of the parameter(s) for the considered individual may be continuous.

Like for the baseline situation, the duration of MP may be one day, one week, or preferably, one month.

At the end of MP, the prevalence of the measured parameter(s) is computed.

The next step 14 of the method comprises comparing the individual's head movement behavior during MP with the baseline situation. To this end, the prevalence of the parameter(s) measured during MP at the measuring step 12 is compared with the prevalence of the same parameter(s) computed at the end of the baseline arbitrary period and obtained at the providing step 10.

In embodiments where each one of a plurality of subareas comprises a different percentage of all head movements for which the parameter(s) is/are measured, the comparing step 14 is performed for each of the subareas.

Any noticeable change from the baseline situation in any area or subarea shall be considered as suspicious i.e. as a sign of early glaucoma or any other above-mentioned eye disease.

Thus, in the following step 16, it is determined whether the comparing step 14 results in detecting a difference between the individual's head movement behavior during MP and the baseline situation and, in case of detecting a difference, it is checked at a following step 18 whether there is any specific reason in the individual's daily life explaining that difference.

For instance, if the detected difference represents a change that is related to a new activity of the individual, involving specific and unusual head movement behavior, it will be categorized as “normal” and/or specific to that new activity.

On the other hand, if there is no specific reason explaining the detected difference, the last step 20 of the method comprises obtaining, on the basis of that difference, information related to a purported evolution of the visual field that may be a forerunner of an eye disease. The change in the individual's head movement behavior constitutes such information.

In an embodiment, optionally, as shown in dashed lined in the flow diagram of FIG. 1, if, at the checking step 18, no specific reason explaining the detected difference is identified, the method according to the present disclosure may further comprise, after the obtaining step 20, a step 22 of generating an alert advising the individual to have eyes checked by an ECP.

In that embodiment, optionally, a predetermined threshold may be set for the difference between the individual's head movement behavior during MP and the baseline situation and the alert generating step 22 is performed only if the difference exceeds the threshold.

The threshold may be determined on the basis of previous studies involving people suffering from one of the above-mentioned eye diseases and unaffected people. It may also be adjusted depending on the individual's profile. For instance, for a person considered as presenting a specific risk, a threshold with higher sensitivity may be chosen.

In an embodiment, the alert may be dispatched to the concerned individual through a mobile communication device, such as a smartphone or tablet or other connected device including a computation unit and an information display unit.

Most common eye diseases are asymmetric, with one eye being affected earlier and more badly than the other. For instance, most of the time, glaucoma affects one eye before the other one and starts by altering the far peripheral visual field. This monocular asymmetry explains why perceptual binocular changes get consciously noticeable only very late in the disease progression. In advanced stages of glaucoma, the areas of monocular field defects coincide spatially and thus result in binocular field loss which is noticed by the patient. The central visual field and the central visual acuity are usually spared, even up to end-stage glaucoma.

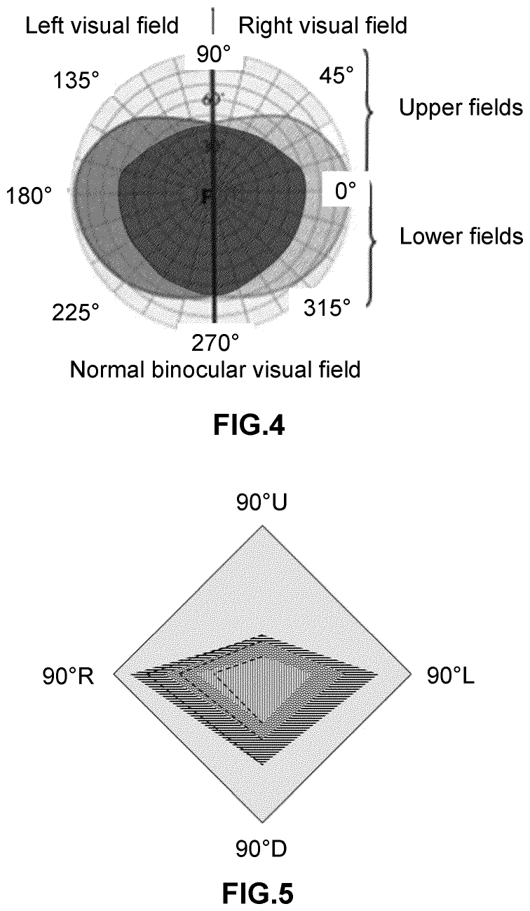

In this respect, FIG. 4 shows overlaps and differences of left and right visual fields. The binocular visual field is shown in the darkest area in the center of the graph. It is composed of the overlap of the right and left monocular visual fields respectively shown on the right lighter part and on the left lighter part of the graph.

If the left eye loses its peripheral visual field earlier than the right eye, the patient has to make more head movements to the left than usually to compensate this loss.

In an embodiment, the detecting step 16 comprises determining whether head movements have changed in one hemi-field and not in the other hemi-field. Eye examination has especially to be considered by the concerned individual as soon as any change in the symmetry of the left and right hemi-fields is detected and if no specific reason for that change is identified at the checking step 18.

A non-limiting example of an outcome of such determining is shown in the graph of FIG. 5, which illustrates an evolution of head movements with respect to those represented in the example of FIG. 3.

It can be observed that there has been an increase by 10 degrees of the rotation amplitude of head movements only to the left compared to the previous situation, in FIG. 3, where both hemi-graphs were symmetric with respect to one another.

In an embodiment, extra weighting may be given to a change in horizontal head rotation with respect to a change in vertical head rotation. For instance, people driving a vehicle frequently need to check whether any other vehicle is coming from the right or left and therefore, at the detecting step 16, a change in the right-left head rotation caused by early glaucoma or by any other above-mentioned eye disease is easier to detect than a change in up-down i.e. vertical head rotation.

More generally, in embodiments, the method according to the present disclosure further comprises, in case of detecting a difference between the individual's head movement behavior during MP and the baseline situation, a step of assigning a different weight to that detected difference, the value of the weight depending on the direction of the head movements.

In a particular embodiment, the method according to the disclosure is computer-implemented. Namely, a computer program product comprises one or more sequences of instructions that are accessible to a processor and that, when executed by the processor, cause the processor to carry out steps of the method as described above for obtaining information related to a purported evolution of the visual field of an individual that may be a forerunner of an eye disease.

The sequence(s) of instructions may be stored in one or several non-transitory computer-readable storage medium/media, including a predetermined location in a cloud.

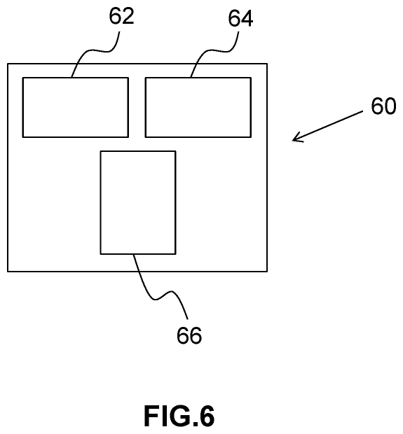

As shown in FIG. 6, a device 60 according to the present disclosure, for obtaining information related to a purported evolution of a visual field of an individual that may be a forerunner of an eye disease, comprises a unit 62 for obtaining a plurality of values of at least one parameter related to head movements, representing a reference head movement behavior.

The device 60 also comprises at least one sensor 64 configured for measuring a same at least one parameter related to head movements of the individual in daily life, during a predetermined measurement period, so as to obtain a plurality of measurements of the at least one parameter, representing the individual's head movement behavior during the predetermined measurement period.

The device 60 further comprises at least one processor 66 configured for:
comparing the individual's head movement behavior during the predetermined measurement period with the reference head movement behavior;
in case of detecting a difference between the individual's head movement behavior during the predetermined measurement period and the reference head movement behavior:
   checking whether there is any specific reason in the individual's daily life explaining that difference;
if there is no such specific reason, obtaining, on the basis of that difference, information related to a purported evolution of the visual field that may be a forerunner of an eye disease.

By way of non-limiting example, the device 60 may be a pair of smart eyeglasses and the at least one sensor 64 may comprise at least one gyroscope and at least one accelerometer. The head movements of the considered individual are recorded by the sensor(s) 64 when the pair of eyeglasses is worn by that individual.

In such an example, the unit 62 may be the at least one sensor 64.

In another example, in a situation where there is a waiting period for the considered individual between the ordering of the smart eyeglasses and their delivery, the unit 62 may be one or more sensors located somewhere else than in/on the smart eyeglasses, e.g. clipped on “regular” (i.e. not smart) eyeglasses, or embedded within a cap or headband or other device worn by the individual. Such devices may be loaned during the waiting period. The measurements made during that period may then be integrated in the baseline measurements or be used as separate baseline measurements.

The sensor(s) 64 does/do not provide absolute measurements of head rotation movements, but relative measurements. For example, a gyroscopic sensor 64 will provide the amount of head rotation between two head movements, but will not provide for instance information regarding the rotation of the head with respect to the torso of the individual.

In order to check symmetry as described above, a head posture absolute reference is advantageously used. This may be done for example by assuming that head rotation movements, when averaged over time, are globally equal to zero i.e. the overall rotation of the head with respect to the torso is null.

Denoting RotAbs(t) the absolute rotation at time instant t, RotRelative(t) the relative rotation at time instant t, Ro a constant and Mean(v) the mean value of a variable v, we have RotAbs(t)=RotRelative(t)+Ro, where Ro is such that Mean(RotAbs(tᵢ,), i=1, . . . , n)=0, where i=1, . . . , n on MP.

In addition, the device 60 may optionally comprise one or more light and/or GPS sensors. This is particularly advantageous in an embodiment where different weights are assigned to the horizontal and vertical head movements, as described above in relationship with the method according to the disclosure. By way of non-limiting example, if the individual is driving, such weighting might be done in an automated manner, by using data from a GPS tracker comprised in the automotive vehicle, for instance.

The present disclosure makes it possible not only to help detecting glaucoma and other retinal pathologies earlier than with prior art clinic tools, but also to follow the evolution of the disease and the effectiveness of the treatment more precisely than prior art subjective assessment methods, which often result in biased or inconsistent decisions.

Although representative systems and methods have been described in detail herein, those skilled in the art will recognize that various substitutions and modifications may be made without departing from the scope of what is described and defined by the appended claims.

1. A method for obtaining information related to a purported evolution of a visual field of an individual, the method comprising:
providing a plurality of values of at least one parameter related to head movements, representing a reference head movement behavior;
measuring a same at least one parameter related to head movements of said individual in daily life, during a predetermined measurement period, so as to obtain a plurality of measurements of said at least one parameter, representing said individual's head movement behavior during said predetermined measurement period;
comparing said individual's head movement behavior during said predetermined measurement period with said reference head movement behavior; and
in case of detecting a difference between said individual's head movement behavior during said predetermined measurement period and said reference head movement behavior:
checking whether there is any specific reason in said individual's daily life explaining said difference; and
if there is no said specific reason, obtaining, on the basis of said difference, information related to a purported evolution of said visual field.

2. The method according to claim 1, wherein said providing comprises generating a statistical model of head movements taking account of at least one datum related to said individual.

3. The method according to claim 2, wherein:
said generating comprises:
measuring said at least one parameter related to head movements of said individual in daily life, during a reference time period equal to a predetermined number of times said measurement period, so as to obtain a plurality of measurements of said at least one parameter, representing said individual's head movement behavior during said reference time period; and
computing, at an end of said reference time period, a prevalence of said at least one parameter related to head movements of said individual in daily life during said reference time period, to be used as said statistical model for said individual;
wherein said method further comprises computing, at an end of said predetermined measurement period, a prevalence of said at least one parameter related to head movements of said individual in daily life during said predetermined measurement period; and
wherein said comparing comprises comparing said prevalence computed at the end of said predetermined measurement period with said prevalence computed at the end of said reference time period.

4. The method according to claim 3, wherein said generating comprises determining said reference time period, by:
(vi) measuring, during a first time period equal to said measurement period, said at least one parameter related to head movements of said individual in daily life, so as to obtain a first plurality of measurements of said at least one parameter;
(vii) measuring, during a second time period equal to said measurement period, said at least one parameter related to head movements of said individual in daily life, so as to obtain a second plurality of measurements of said at least one parameter;
(viii) determining whether there are differences considered as significant between said first and second pluralities of measurements;
(ix) in case there is no difference considered as significant between said first and second pluralities of measurements, determining said reference time period as said measurement period;
(x) in case there are differences considered as significant between said first and second pluralities of measurements:
(d) measuring, during an additional time period equal to said measurement period, said at least one parameter related to head movements of said individual in daily life, so as to obtain an additional plurality of measurements;
(e) determining whether there are differences considered as significant between, on the one hand, cumulated pluralities of measurements obtained at steps (i) and (ii) and, on the other hand, said additional plurality of measurements; and
(f) iterating step (a) during further time periods, each equal to said measurement period, as long as there are differences considered as significant between pluralities of measurements used at step (b), so as to eventually determine said reference time period as an accumulation of time periods of steps (i), (ii), (a) and (c).

5. The method according to claim 1, wherein said at least one parameter is taken in a group comprising a rotation amplitude of each one of said head movements, a rotation speed of each one of said head movements and a variance of rotation speed of said head movements.

6. The method according to claim 1, further comprising, if there is no said specific reason, generating an alert advising said individual to have eyes checked by an eye care professional.

7. The method according to claim 6, wherein said generating said alert is performed only if said difference between said individual's head movement behavior during said predetermined measurement period and said reference head movement behavior exceeds a predetermined threshold.

8. The method according to claim 1, wherein it further comprises further comprising, in case of detecting a difference between said individual's head movement behavior during said predetermined measurement period and said reference head movement behavior, assigning a different weight to said difference depending on a direction of head movements.

9. The method according to claim 1, wherein said detecting comprises determining whether head movements have changed in one hemi-field and not in another hemi-field.

10. The method according to claim 1, further comprising:
dividing an area comprising all possible head movements into a plurality of subareas, each of said subareas comprising a different predetermined percentage of all head movements for which said at least one parameter is measured during said measuring; and
performing for each of said subareas said comparing said individual's head movement behavior during said predetermined measurement period with said reference head movement behavior.

11. The method according to claim 1, wherein said measuring is continuous.

12. A device for obtaining information related to a purported evolution of a visual field of an individual, the device comprising:
a unit for obtaining a plurality of values of at least one parameter related to head movements, representing a reference head movement behavior;
at least one sensor configured for measuring a same at least one parameter related to head movements of said individual in daily life, during a predetermined measurement period, so as to obtain a plurality of measurements of said at least one parameter, representing said individual's head movement behavior during said predetermined measurement period; and
at least one processor configured for:
comparing said individual's head movement behavior during said predetermined measurement period with said reference head movement behavior; and
in case of detecting a difference between said individual's head movement behavior during said predetermined measurement period and said reference head movement behavior:
checking whether there is any specific reason in said individual's daily life explaining said difference; and
if there is no said specific reason, obtaining, on the basis of said difference, information related to a purported evolution of said visual field.

13. The device according to claim 12, wherein said device is a pair of smart eyeglasses and said at least one sensor comprises at least one gyroscope and at least one accelerometer.

14. A computer program product comprising one or more sequences of instructions that are accessible to a processor and that, when executed by said processor, cause said processor to:
obtain a plurality of values of at least one parameter related to head movements, representing a reference head movement behavior;
receive a plurality of measurements of a same at least one parameter related to head movements of an individual in daily life, during a predetermined measurement period, representing said individual's head movement behavior during said predetermined measurement period;
compare said individual's head movement behavior during said predetermined measurement period with said reference head movement behavior; and
in case of detecting a difference between said individual's head movement behavior during said predetermined measurement period and said reference head movement behavior:
check whether there is any specific reason in said individual's daily life explaining said difference; and
if there is no said specific reason, obtain, on the basis of said difference, information related to a purported evolution of a visual field of said individual.

15. A non-transitory information storage medium, wherein it stores one or more sequences of instructions that are accessible to a processor and that, when executed by said processor, cause said processor to:
obtain a plurality of values of at least one parameter related to head movements, representing a reference head movement behavior;
receive a plurality of measurements of a same at least one parameter related to head movements of an individual in daily life, during a predetermined measurement period, representing said individual's head movement behavior during said predetermined measurement period;
compare said individual's head movement behavior during said predetermined measurement period with said reference head movement behavior; and
in case of detecting a difference between said individual's head movement behavior during said predetermined measurement period and said reference head movement behavior:
check whether there is any specific reason in said individual's daily life explaining said difference; and
if there is no said specific reason, obtain, on the basis of said difference, information related to a purported evolution of a visual field of said individual.
